(19) United States
(12) Patent Application Publication
(10) Pub. No.: US 2002/0115162 A1
(43) Pub. Date: Aug. 22, 2002

(54) Nucleotide sequences coding for the cysQ gene

(76) Inventors: Mike Farwick, Bielefeld (DE); Klaus Huthmacher, Gelnhausen (DE); Brigitte Bathe, Salzkotten (DE); Walter Pfefferle, Halle (DE)

Correspondence Address: SMITH, GAMBRELL & RUSSELL, LLP, Suite 800, 1850 M Street, N.W., Washington, DC, 20036, US

(21) Appl. No.: 09/987,446

(22) Filed: Nov. 14, 2001

(30) Foreign Application Priority Data
Nov. 22, 2000 (DE) DE 100 57 801.2

(51) Int. Cl. C12P 13/08; C07H 21/02; C07H 21/04; C12P 21/02; C12N 1/21; C07K 14/195

(52) U.S. Cl. 435/115; 435/69.1; 435/252.3; 435/320.1; 530/350; 536/23.1

(57) ABSTRACT

The invention relates to polynucleotide sequences from coryneform bacteria coding for the cysQ gene and a process for the fermentative preparation of amino acids using bacteria in which the endogenous cysQ gene is enhanced, as well as to the use of polynucleotides containing the sequences according to the invention as hybridization probes.

### BACKGROUND OF THE INVENTION

The invention provides nucleotide sequences from coryneform bacteria coding for the cysQ gene and a process for the fermentative preparation of amino acids using bacteria in which the endogenous cysQ gene is enhanced. All references cited herein are expressly incorporated by reference. Incorporation by reference is also designated by the term “I.B.R.” following any citation.

L-amino acids, particularly L-lysine, L-cysteine and L-methionine, are used in human medicine and in the pharmaceutical industry, the food industry and more particularly in animal nutrition.

It is known that amino acids are prepared by fermentation of strains of coryneform bacteria, particularly Corynebacterium glutamicum. In view of its great importance, work is constantly being carried out to improve the preparation processes. Process improvements may relate to measures involving the fermentation technique such as, for example, agitation and oxygen supply, or the composition of the nutrient media such as, for example, the sugar concentration during fermentation, or the workup to the product form by, for example, ion exchange chromatography, or the intrinsic performance properties of the microorganism itself.

In order to improve the performance properties of said microorganisms, methods of mutagenesis, selection and mutant selection are employed. Strains thereby obtained are resistant to antimetabolites or auxotrophic for metabolites of regulatory importance and produce amino acids.

For some years, methods of recombinant DNA technology have also been used to improve strains of Corynebacterium producing L-amino acids by amplifying individual amino acid biosynthesis genes and examining the effect on amino acid production.

The invention provides new measures for the improved fermentative preparation of amino acids.

### BRIEF SUMMARY OF THE INVENTION

Where the terms L-amino acids or amino acids are mentioned below, they refer to one or more amino acids including the salts thereof, selected from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine and the sulfur-containing L-amino acids L-cysteine and L-methionine are particularly preferred.

Where the terms L-lysine or lysine are mentioned below, they refer not only to the bases but also to the salts such as, e.g., lysine monchydrochloride or lysine sulfate.

Where the terms L-cysteine or cysteine are mentioned below, they also refer to the salts such as, e.g., cysteine hydrochloride or cysteine-S-sulfate.

Where the terms L-methionine or methionine are mentioned below, they also refer to the salts such as, e.g., methionine hydrochloride or methionine sulfate.

The invention provides an isolated polynucleotide from coryneform bacteria containing a polynucleotide sequence coding for the cysQ gene, selected from the group consisting of

a) polynucleotide which is at least 70% identical to a polynucleotide coding for a polypeptide which contains the amino acid sequence of SEQ ID no. 2,

b) polynucleotide which codes for a polypeptide containing an amino acid sequence which is at least 70% identical to the amino acid sequence of SEQ ID no. 2,

c) polynucleotide which is complementary to the polynucleotides of a) or b), and

d) polynucleotide containing at least 15 successive nucleotides of the polynucleotide sequence of a), b) or c),

wherein the polypeptide preferably has the activity of the transport protein CysQ.

The invention also provides the above-mentioned polynucleotide, this being preferably a replicable DNA containing:

(i) the nucleotide sequence shown in SEQ ID no. 1, or

(ii) at least one sequence which corresponds to the sequence(i) within the degeneracy region of the genetic code, or

(iii) at least one sequence which hybridises with the sequence complementary to sequence (i) or (ii), and optionally

(iv) functionally neutral sense mutations in (i) which do not alter the activity of the protein/polypeptide.

Finally, the invention also provides polynucleotides selected from the group consisting of

a) polynucleotides containing at least 15 successive nucleotides selected from the nucleotide sequence of SEQ ID no. 1 between the positions 1 and 1013,

b) polynucleotides containing at least 15 successive nucleotides selected from the nucleotide sequence of SEQ ID no. 1 between the positions 1014 and 1769, and

c) polynucleotides containing at least 15 successive nucleotides selected from the nucleotide sequence of SEQ ID no. 1 between the positions 1770 and 2730.

The invention also provides

a replicable polynucleotide, particularly DNA, containing the nucleotide sequence as shown in SEQ ID no. 1;

a polynucleotide coding for a polypeptide which contains the amino acid sequence as shown in SEQ ID no. 2;

a vector containing the polynucleotide according to the invention, particularly shuttle vector or plasmid vector, and

coryneform bacteria which contain the vector or in which the endogenous cysQ gene is enhanced.

The invention also provides polynucleotides comprising substantially a polynucleotide sequence which may be obtained by screening by hybridising an appropriate gene library of a coryneform bacterium which contains the complete gene or parts thereof, with a probe which contains the sequence of the polynucleotide according to the invention according to SEQ ID no.1 or a fragment thereof, and isolating the polynucleotide sequence mentioned.

### DETAILED DESCRIPTION OF THE INVENTION

Polynucleotides containing the sequences according to the invention are suitable as hybridization probes for RNA, cDNA and DNA in order to isolate nucleic acids or polynucleotides or genes in their full length which code for the transport protein CysQ, or in order to isolate those nucleic acids or polynucleotides or genes which have great similarity of sequence with that of the cysQ gene. They may also be deposited as a probe on arrays, micro arrays or DNA chips in order to detect and determine the corresponding polynucleotides or sequences derived therefrom such as, e.g., RNA or cDNA.

Polynucleotides containing the sequences according to the invention are also suitable as primers for the preparation of DNA of genes coding for the transport protein CysQ by means of the polymerase chain reaction (PCR).

The oligonucleotides acting as probes or primers contain at least 25, 26, 27, 28, 29 or 30, preferably at least 20, 21, 22, 23 or 24, more particularly preferably at least 15, 16, 17, 18 or 19 successive nucleotides. Oligonucleotides with a length of at least 31, 32, 33, 34, 35, 36, 37, 38, 39 or 40, or at least 41, 42, 43, 44, 4S, 46, 47, 48, 49 or 50 nucleotides are also suitable. Optionally, oligonucleotides with a length of at least 100, 150, 200, 250 or 300 nucleotides are also suitable.

“Isolated” means separated from its natural surroundings.

“Polynucleotide” refers generally to polyribonucleotides and polydeoxyribonucleotides which may be unmodified RNA or DNA or modified RNA or DNA.

The polynucleotides according to the invention include a polynucleotide according to SEQ ID no. 1 or a fragment prepared therefrom, and also those which are at least particularly 70% to 80%, preferably at least 81% to 85%, particularly preferably at least 86% to 90% and more particularly preferably at least 91%, 93%, 95%, 97% or 99% identical to the polynucleotide according to SEQ ID no. 1 or a fragment prepared therefrom.

The term “polypeptides” means peptides or proteins which contain two or more amino acids bound by way of peptide bonds.

The polypeptides according to the invention include a polypeptide according to SEQ ID no. 2, particularly those with the biological activity of the transport protein CysQ and also those which are at least 70% to 80%, preferably at least 81% to 85%, particularly preferably at least 86% to 90%, and more particularly preferably at least 91%, 93%, 95%, 97% or 99% identical to the polypeptide according to SEQ ID no. 2 and have the activity mentioned.

The invention also relates to a process for the fermentative preparation of amino acids selected from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using coryneform bacteria which, in particular, already produce amino acids and in which the nucleotide sequences coding for the cysQ gene are enhanced, particularly overexpressed.

The term “enhancement” describes in this connection increasing the intracellular activity of one or more enzymes in a microorganism which are coded for by the corresponding DNA by, for example, increasing the copy number of the gene or genes, using a strong promoter or using a gene which codes for a corresponding enzyme with a high activity, and optionally combining these measures.

As a result of the enhancement measures, particularly overexpression, the activity or concentration of the corresponding protein is generally increased by at least 10%, 25%, 50%, 75%, 100%, 150%, 200%, 300%, 400% or 500%, by a maximum of up to 1,000% or 2,000% relative to that of the wild-type protein, or the activity or concentration of the protein in the starting microorganism.

The microorganisms which are the subject of the present invention may produce L-amino acids from glucose, sucrose, lactose, fructose, maltose, molasses, starch, cellulose or from glycerol and ethanol. They may be representatives of coryneform bacteria, particularly of the Corynebacterium genus. A particular example of the Corynebacterium genus is the Corynebacterium glutamicum type which is known by skilled persons for its ability to produce L-amino acids.

Examples of suitable strains of the Corynebacterium genus, particularly of the Corynebacterium glutamicum type (C. glutamicum) include, in particular, the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-amino acid-producing mutants or strains prepared therefrom.

The new cysQ gene of C. glutamicum coding for the transport protein CysQ was isolated.

In order to isolate the cysQ gene or other genes from C. glutamicum, a gene library of this microorganism is first prepared in Escherichia coli (E. coli). The preparation of gene libraries is documented in generally known textbooks and manuals. Examples include the textbook by Winnacker: Gene und Klone, Eine Einführung in die Gentechnologie (Verlag Chemie, Weinheim, Germany, 1990) I.B.R., or the manual by Sambrook et al.: Molecular Cloning, A Laboratory Manual (Cold Spring Harbor Laboratory Press, 1989) I.B.R. A very well known gene library is that of the E. coli K-12 strain W3110, which was prepared by Kohara et al. (Cell 50, 495-508 (1987) I.E.R.) in λ-vectors. Bathe et al. (Molecular and General Genetics, 252:255-265, 1996) I.B.R. describe a gene library of C. glutamicum ATCC13032, which was prepared using the cosmid vector SuperCos I (Wahl et al., 1987, Proceedings of the National Academy of Sciences USA, 84:2160-2164 I.D.R.) in the E. coli K-12 strain NM554 (Raleigh et al., 1988, Nucleic Acids Research 16:1563-1575 I.B.R.).

Börmann et al. (Molecular Microbiology 6(3), 317-326 (1992)) I.B.R. in turn describe a gene library of C. glutamicum ATCC13032 using the cosmid pHC79 (Hohn and Collins, 1980, Gene 11, 291-298 I.B.R.).

In order to prepare a gene library of C. glutamicum in E. coli, it is also possible to use plasmids such as pBR322 (Bolivar, 1979, Life Sciences, 25, 807-818 I.B.R.) or pUC9 (Vieira et al., 1982, Gene, 19:259-268 I.B.R.). Particularly suitable hosts are E. coli strains which are restriction- and recombination-defective. An example hereof is the DH5αmcr strain which was described by Grant et al. (Proceedings of the National Academy of Sciences USA, 87 (1990) 4645-4649) I.B.R. The long DNA fragments cloned using cosmids may then in turn be subcloned into common vectors suitable for sequencing and then sequenced, as described, e.g., by Sanger et al. (Proceedings of the National Academy of Sciences of the United States of America, 74:5463-5467, 1977) I.B.R.

The DNA sequences obtained may then be analyzed with known algorithms or sequence analysis programs such as, e.g. that of Staden (Nucleic Acids Research 14, 217-232(1986) I.B.R.), that of Marck (Nucleic Acids Research 16, 1829-1836 (1988) I.B.R.) or the GCG program of Butler (Methods of Biochemical Analysis 39, 74-97 (1998)) I.B.R.

The new DNA sequence coding for the cysQ gene of C. glutamicum was found which, as SEQ ID no. 1, forms part of the present invention. Moreover, the amino acid sequence of the corresponding protein was derived from the DNA sequence in question with the methods described above. The resulting amino acid sequence of the cysQ gene product is shown in SEQ ID no. 2. It is known that enzymes belonging to the host are able to cleave the N-terminal amino acid methionine or formylmethionine of the protein formed.

Coding DNA sequences resulting from SEQ ID no. 1 due to the degeneracy of the genetic code also form part of the invention. In the same way, DNA sequences which hybridize with SEQ ID no. 1 or parts of SEQ ID no. 1, form part of the invention. Experts are also familiar with conservative amino acid exchanges such as, e.g., the exchange of glycine for alanine or of aspartic acid for glutamic acid in proteins as sense mutations which do not lead to a fundamental change in the activity of the protein, i.e. which are functionally neutral. Mutations of this kind are also known, inter alia, as neutral substitutions. It is also known that changes at the N and/or C end of a protein do not substantially impair or may even stabilize its function. Skilled persons may find details on this subject, inter alia, in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in well known textbooks of genetics and molecular biology. Amino acid sequences which are obtained in a corresponding manner from SEQ ID no. 2 also form part of the invention.

In the same way, DNA sequences which hybridize with SEQ ID no. 1 or parts of SEQ ID no. 1 form part of the invention. Finally, DNA sequences which are prepared by the polymerase chain reaction (PCR) using primers obtained from SEQ ID no. 1 form part of the invention. Such oligonucleotides typically have a length of at least 15 nucleotides.

The skilled person may find instructions for the identification of DNA sequences by hybridization inter alia in the manual “The DIG System Users Guide for Filter Hybridisation” by Boehringer Mannheim GmbH (Mannheim, Germany, 1993) I.B.R. and in Liebl et al. (International Journal of Systematic Bacteriology 41: 255-260 (1991)) I.B.R. Hybridization takes place under stringent conditions, that is, only hybrids are formed in which probe and target sequence, i.e. the polynucleotides treated with the probe, are at least 70% identical. It is known that the stringency of hybridization including the wash steps is affected or determined by varying the buffer composition, temperature and salt concentration. The hybridization reaction is carried out preferably with relatively low stringency compared with the wash steps (Hybaid Hybridisation Guide, Hybaid Limited, Teddington, UK, 1996) I.B.R.

For example a 5× SSC buffer may be used at a temperature of about 50° C.-68° C. for the hybridization reaction. In this case, probes may also hybridize with polynucleotides that are less than 70% identical to the sequence of the probe. Such hybrids are less stable and are removed by washing under stringent conditions. This may be achieved, for example, by reducing the salt concentration to 2 SSC and optionally subsequently 0.5× SSC (The DIG System User's Guide for Filter Hybridisation, Boehringer Mannheim, Mannheim, Germany, 1995 I.B.R.), a temperature of about 50° C.-68° C. being obtained. It is also possible, optionally, to reduce the salt concentration to as low as 0.1× SSC. By raising the hybridization temperature stepwise from 50° C. to 68° C. in steps of about 1-2° C., polynucleotide fragments can be isolated which are, for example, at least 70% or at least 80% or at least 90% to 95% identical to the sequence of the probe used. Further instructions on hybridization are available commercially in the form of kits(e.g. DIG Easy Hyb from Roche Diagnostics GmbH, Mannheim, Germany, Catalogue No. 1603558).

The skilled person may find instructions for the amplification of DNA sequences using the polymerase chain reaction (PCR) inter alia in the manual by Gait: Oligonucleotide Synthesis: A Practical Approach (IRL Press, Oxford, UK, 1984) I.B.R. and in Newton and Graham: PCR (Spektrum Akademischer Verlag, Heidelberg, Germany, 1994 I.B.R).

It has been found that coryneform bacteria produce amino acids in an improved manner after overexpression of the cysQ gene.

In order to obtain overexpression, the copy number of the corresponding genes may be increased, or the promoter and regulatory region or the ribosome binding site situated upstream from the structural gene may be mutated. Expression cassettes incorporated upstream from the structural gene act in the same way. By means of inducible promoters it is also possible to increase expression during the course of fermentative amino acid production. Expression is also improved by measures to prolong the life of the m-RNA. Moreover, the enzyme activity is also enhanced by preventing degradation of the enzyme protein. The genes or gene constructs may either be present in plasmids with a different copy number or integrated in the chromosome and amplified. Alternatively, moreover, overexpression of the genes concerned may be achieved by altering the composition of the medium and the culture method.

The skilled person will find instructions on this matter, inter alia, in Martin et al. (Bio/Technology 5, 137-146 (1987)) I.B.R., in Guerrero et al. (Gene 138, 35-41 (1994)) I.D.R., Tsuchiya and Morinaga (Bio/Technology 6, 428-430 (1988)) I.B.R., in Eikmanns et al. (Gene 102, 93-98 (1991)) I.B.R., in European patent 0 472 869 I.B.R., in U.S. Pat. No. 4,601,893 I.B.R., in Schwarzer and Pühler (Bio/Technology 9, 84-87 (1991) I.B.R., in Reinscheid et al. (Applied and Environmental Microbiology 60, 126-132 (1994)) I.B.R., in LaBarre et al. (Journal of Bacteriology 175, 1001-1007 (1993)) I.B.R., in patent application WO 96/15246 I.B.R., in Malumbres et al. (Gene 134, 15-24 (1993)) I.E.R., in Japanese published patent application JP-A-10-229891 I.B.R., in Jensen and Hammer (Biotechnology and Bioengineering 58, 191-195 (1998)) I.B.R., in Makrides (Microbiological Reviews 60:512-538 (1996)) I.B.R. and in known textbooks of genetics and molecular biology.

For the purpose of enhancement, the cysQ gene according to the invention was overexpressed by way of example with episomal plasmids. Suitable plasmids are those which are replicated in coryneform bacteria. Numerous well known plasmid vectors such as, e.g., pZl (Menkel et al., Applied and Environmental Microbiology (1989) 64: 549-554 I.B.R.), pEKEx1 (Eikmanns et al., Gene 102:93-98 (1991 I.B.R.)) or pHS2-1 (Sonnen et al., Gene 107:69-74 (1991) I.B.R.) are based on the cryptic plasmids pHM1519, pBL1 or pGA1. Other plasmid vectors such as, e.g., those based on pCG4(U.S. Pat. No. 4,489,160) I.E.R., or pNG2 (Serwold-Davis et al., FEMS Microbiology Letters 66, 119-124 (1990) I.E.R.), or pAG1(U.S. Pat. No. 5,158,891 I.B.R.), may be used in the same way.

Further suitable plasmid vectors are those by means of which the process of gene amplification by integration into the chromosome may be used, as described, for example, by Reinscheid et al. (Applied and Environmental Microbiology 60, 126-132 (1994) I.B.R.) for the duplication and amplification of the hom-thrE operon. In this method, the complete gene is cloned into a plasmid vector which is able to replicate in a host (typically E. coli), but not in C. glutamicum. Suitable vectors include, for example, pSUP301 (Simon et al., Bio/Technology 1, 784-791 (1983) I.B.R.), pK18mob or pK19mob (Schäfer et al., Gene 145, 69-73 (1994) I.B.R.), PGEM-T (Promega Corporation, Madison, Wis., USA), pCR2.1-TOPO (Shuman (1994). Journal of Biological Chemistry 269:32678-84 I.B.R.; U.S. Pat. No. 5,487,993 I.B.R.), pCR®Blunt (Firma Invitrogen, Groningen, Netherlands; Bernard et al., Journal of Molecular Biology, 234: 534-541 (1993) I.B.R.), pEM1 (Schrumpf et al, 1991, Journal of Bacteriology 173:4510-4516 I.B.R.) or PBGS8 (Spratt et al.,1986, Gene 41: 337-342 I.B.R.). The plasmid vector containing the gene to be amplified is then transferred by conjugation or transformation into the desired strain of C. glutamicum. The method of conjugation is described, for example, in Schäfer et al. (Applied and Environmental Microbiology 60, 756-759 (1994)) I.B.R. Methods of transformation are described, for example, in Thierbach et al. (Applied Microbiology and Biotechnology 29, 356-362 (1988) I.B.R.), Dunican and Shivnan (Bio/Technology 7, 1067-1070 (1989) I.B.R.) and Tauch et al. (FEMS Microbiological Letters 123, 343-347 (1994)) I.B.R. After homologous recombination by means of a cross-over event, the resulting strain contains at least two copies of the gene in question.

In addition, it may be advantageous for the production of L-amino acids, in addition to enhancing the cysQ gene, to enhance, particularly to overexpress, one or more enzymes of the biosynthetic pathway in question, glycolysis, anaplerotic reaction, the citric acid cycle, the pentose phosphate cycle, amino acid export and optionally regulatory proteins.

For the preparation of L-amino acids, it is possible in addition to enhancing the cysQ gene, to enhance, particularly to overexpress, one or more endogenous genes, according to the biosynthetic pathway, selected from the group comprising

the dapA gene coding for dihydrodipicolinate synthase (EP-B 0 197 335 I.B.R.),

the gap gene coding for glyceraldehyde-3-phosphate dehydrogenase (Eikmanns (1992), Journal of Bacteriology 174:6076-6086 I.B.R.),

the tpi gene coding for triosephosphate isomerase (Eikmanns (1992), Journal of Bacteriology 174:6076-6086

the pgk gene coding for 3-phosphoglycerate kinase (Eikmanns (1992), Journal of Bacteriology 174:6076-6086 I.B.R.),

the zwf gene coding for glucose-6-phosphate dehydrogenase (JP-A-09224661 I.B.R.),

the pyc gene coding for pyruvate carboxylase (DE-A-198 31 609 I.B.R.),

the mqo gene coding for malate quinone oxidoreductase (Molenaar et al., European Journal of Biochemistry 254, 395-403 (1998) I.B.R.),

the lysC gene coding for a feedback resistant aspartate kinase (Accession No.P26512),

the lysE gene coding for lysine export (DE-A-195 48 222 I.B.R.),

the hom gene coding for homoserin dehydrogenase (EP-A 0131171 I.B.R.),

the ilvA gene coding for threonine dehydratase (Möckel et al., Journal of Bacteriology (1992) 8065-8072) I.B.R.) or the ilvA(Fbr) allele coding for a feedback resistant threonine dehydratase (Mbckel et al., (1994) Molecular Microbiology 13: 833-842 I.B.R.),

the ilvBN gene coding for acetohydroxy acid synthase (EP-B 0356739 I.B.R.),

the ilvD gene coding for dihydroxy acid dehydratase (Sahm and Eggeling (1999) Applied and Environmental Microbiology 65: 1973-1979 I.B.R.),

the zwal gene coding for the Zwal protein (DE: 19959328.0 I.B.R., DSM 13115).

Moreover, for the production of L-amino acids, it may be advantageous, in addition to enhancing the cysQ gene, to attenuate one or more genes selected from the group comprising

the pck gene coding for phosphoenolpyruvate carboxykinase (DE 199 50 409.1 I.B.R., DSM 13047),

the pgi gene coding for glucose-6-phosphate isomerase (U.S. Pat. No. 09/396,478 I.B.R., DSM 12969),

the poxB gene coding for pyruvate oxidase (DE:1995 1975.7 I.B.R., DSM 13114),

the zwa2 gene coding for the Zwa2 protein (DE: 19959327.2 I.B.R., DSM 13113)

particularly to reduce the expression.

Moreover, for the production of amino acids, it may be advantageous, in addition to overexpressing the cysQ gene, to exclude unwanted side reactions (Nakayama: “Breeding of Amino Acid Producing Microorganisms”, in: Overproduction of Microbial Products, Krumphanzl, Sikyta, Vanek (eds.), Academic Press, London, UK, 1982 I.B.R.).

The microorganisms produced according to the invention also form part of the invention and may be cultivated continuously or batchwise in the batch process (batch cultivation) or in the fed-batch or repeated fed-batch process in order to produce amino acids. A summary of well known cultivation methods is described in the textbook by Chmiel (Bioprozesstechnik l. Einfuhrung in die Bioverfahrenstechnik (Gustav Fischer Verlag, Stuttgart, 1991) I.B.R.) or in the textbook by Storhas (Bioreaktoren und periphere Einrichtungen (Vieweg Verlag, Braunschweig/Wiesbaden, 1994) I.B.R.).

The culture medium to be used must satisfy the requirements of the strains concerned in a suitable manner. Descriptions of culture media of various microorganisms are contained in the manual “Manual of Methods for General Bacteriology” of the American Society for Bacteriology (Washington D.C., USA, 1981) I.B.R.

Suitable sources of carbon include sugars and carbohydrates such as, e.g., glucose, sucrose, lactose, fructose, maltose, molasses, starch and cellulose, oils and fats such as, e.g., soyabean oil, sunflower oil, groundnut oil and coconut fat, fatty acids such as, e.g., palmitic acid, stearic acid and linoleic acid, alcohols such as, e.g. glycerol and ethanol and organic acids such as, e.g., acetic acid. Said substances may be used individually or as a mixture.

Suitable sources of nitrogen include organic nitrogen-containing compounds such as peptones, yeast extract, meat extract, malt extract, maize swelling water, soyabean flour and urea or inorganic compounds such as ammonium sulfate, ammonium chloride, ammonium phosphate, ammonium carbonate and ammonium nitrate. The sources of nitrogen may be used individually or as a mixture.

Suitable sources of phosphorus include phosphoric acid, potassium dihydrogen phosphate or dipotassium hydrogen phosphate or the corresponding sodium-containing salts. The culture medium must also contain salts of metals such as, e.g., magnesium sulfate or iron sulfate which are necessary for growth. Finally, essential growth-promoters such as amino acids and vitamins may be used in addition to the substances mentioned above. Moreover, suitable precursors may be added to the culture medium. The above-mentioned substances used may be added to the culture in the form of a single preparation or fed in a suitable manner during cultivation.

In order to control the pH of the culture, basic compounds such as sodium hydroxide, potassium hydroxide, ammonia or ammonia solution or acid compounds such as phosphoric acid or sulfuric acid are used in a suitable manner. Anti-foaming agents such as, e.g., fatty acid polyglycol esters may be used to control foam development. In order to maintain the stability of plasmids, suitable selectively acting substances such as, e.g., antibiotics may be added to the medium. In order to maintain aerobic conditions, oxygen or oxygen-containing gas mixtures such as, e.g., air may be introduced into the culture. The temperature of the culture is normally from 20° C. to 45° C. and preferably from 25° C. to 40° C. The culture is continued until a maximum of the desired product has been formed. This objective is normally achieved within 10 hours to 160 hours.

Methods for determining L-amino acids are known from the prior art. The analysis may be carried out, for example, as described in Spackman et al. (Analytical Chemistry, 30, (1958), 1190 I.B.R.) by ion exchange chromatography followed by ninhydrin derivation, or it may take place by reversed phase HPLC as described in Lindroth et al. (Analytical Chemistry (1979) 51: 1167-1174) I.B.R.

The process according to the invention is used for the fermentative preparation of amino acids.

The present invention is explained in more detail below with reference to specific embodiments.

The isolation of plasmid DNA from Escherichia coli and all the methods of restriction, Klenow und alkaline phosphatase treatment were carried out in accordance with Sambrook et al. (Molecular Cloning. A Laboratory Manual (1989) Cold Spring Harbor Laboratory Press, Cold Spring Harbor, N.Y., USA I.B.R.). Methods for the transformation of Escherichia coli are also described in this manual.

The composition of common nutrient media such as LB or TY medium can also be derived from the manual by Sambrook et al.

### EXAMPLE 1

Preparation of a Genomic Cosmid Gene Library from Corynebacterium glutamicum ATCC 13032

Chromosomal DNA from Corynebacterium glutamicum ATCC 13032 was isolated as described in Tauch et al. (1995, Plasmid 33:168-179) I.B.R. and partially cleaved with the restriction enzyme Sau3AI (Amersham Pharmacia, Freiburg, Germany, product description Sau3AI, code no. 27-0913-02). The DNA fragments were dephosphorylated with Shrimp alkaline phosphatase (Roche Diagnostics GmbH, Mannheim, Germany, product description SAP, code no. 1758250). The DNA of the cosmid vector SuperCosl (Wahl et al. (1987) Proceedings of the National Academy of Sciences USA 84:2160-2164 I.B.R.), purchased from Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, code no. 27-0948-02) and likewise dephosphorylated with Shrimp alkaline phosphatase.

The cosmid DNA was then cleaved with the restriction enzyme BamHI (Amersham Pharmacia, Freiburg, Germany, product description BamHI, code no. 27-0868-04). The cosmid DNA treated in this way was mixed with the treated ATCC 13032-DNA and the batch was treated with T4-DNA-ligase (Amersham Pharmacia, Freiburg, Germany, product description T4-DNA-Ligase, code no.27-0870-04). The ligation mixture was then packaged into phages using Gigapack II XL Packing Extracts (Stratagene, La Jolla, USA, product description Gigapack II XL Packing Extract, code no. 200217).

In order to infect the E. coli strain NM554 (Raleigh et al. 1988, Nucleic Acids Research 16:1563-1575 I.B.R.) the cells were taken up in 10 mM MgSO₄ and mixed with an aliquot of the phage suspension. Infection and titering of the cosmid library were carried out as described in Sambrook et al. (1989, Molecular Cloning: A Laboratory Manual, Cold Spring Harbor) I.B.R., the cells being plated on LB agar (Lennox, 1955, Virology, 1:190 I.B.R.) with 100 pg/ml ampicillin. After incubation overnight at 37° C., recombinant individual clones were selected.

### EXAMPLE 2

Isolation and Sequencing of the cysQ Gene

The cosmid DNA of an individual colony was isolated with the Qiaprep Spin Miniprep Kit (product no. 27106, Qiagen, Hilden, Germany) in accordance with the manufacturer's instructions and partially cleaved with the restriction enzyme Sau3AI (Amersham Pharmacia, Freiburg, Germany, product description Sau3AI, product no. 27-0913-02). The DNA fragments were dephosphorylated with Shrimp alkaline phosphatase (Roche Diagnostics GmbH, Mannheim, Germany, product description SAP, product no. 1758250). After separation by gel electrophoresis, isolation of the cosmid fragments in the size range from 1500 to 2000 bp was carried out with the QiaExII Gel Extraction Kit (product no. 20021, Qiagen, Hilden, Germany).

The DNA of the sequencing vector pzero-1 purchased from Invitrogen (Groningen, the Netherlands, product description Zero Background Cloning Kit, product no. K2500-01) was cleaved with the restriction enzyme BamHI (Amersham Pharmacia, Freiburg, Germany, product description BamHI, product no. 27-0868-04). Ligation of the cosmid fragments into the sequencing vector pZero-l was carried out as described by Sambrook et al. (1989, Molecular Cloning: A Laboratory Manual, Cold Spring Harbor) I.B.R., the DNA mixture being incubated overnight with T4-ligase (Pharmacia Biotech, Freiburg, Germany). This ligation mixture was then electroporated into the E. coli strain DH5αMCR (Grant, 1990, Proceedings of the National Academy of Sciences U.S.A., 87:4645-4649 I.B.R.) (Tauch et al. 1994, FEMS Microbiol Letters, 123:343-7 I.B.R.) and plated on LB agar (Lennox, 1955, Virology, 1:190) I.B.R. with 50 mg/l Zeocin.

Plasmid preparation of the recombinant clones was carried out with the Biorobot 9600 (product no. 900200, Qiagen, Hilden, Germany). Sequencing was carried out by the dideoxy-chain termination method of Sanger et al. (1977, Proceedings of the National Academy of Sciences U.S.A., 74:5463-5467 I.B.R.) with modifications according to Zimmermann et al. (1990, Nucleic Acids Research, 18:1067 I.B.R.). The “RR dRhodamin Terminator Cycle Sequencing Kit” from PE Applied Biosystems (product no. 403044, Weiterstadt, Germany) was used. Separation by gel electrophoresis and analysis of the sequencing reaction was carried out in a “Rotiphoresis NF acrylamide/bisacrylamide” gel (29:1) (product no. A124.1, Roth, Karlsruhe, Germany) with the “ABI Prism 377” sequencing device from PE Applied Biosystems (Weiterstadt, Germany).

The raw sequence data obtained were then processed using the Staden program package (1986, Nucleic Acids Research, 14:217-231 I.B.R.) version 97-0. The individual sequences of the pZerol derivatives were assembled to a coherent contig. Further analyses can be carried out with the “BLAST search program” (Altschul et al., 1997, Nucleic Acids Research, 25:3389-3402 I.B.R.) against the non-redundant databank of the “National Center for Biotechnology Information” (NCBI, Bethesda, Md., USA) I.B.R.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The nucleotide sequence obtained is shown in SEQ ID no.1. The analysis of the nucleotide sequence revealed an open reading frame of 759 base pairs, which was designated the cysQ gene. The cysQ gene codes for a protein of 252 amino acids.

This application claims priority to German Priority Document Application No. 100 57 801.2, filed on Nov. 22, 2000. The above German Priority Document is hereby incorporated by reference in its entirety.

We claim:

1. An isolated polynucleotide from coryneform bacteria, containing a polynucleotide sequence coding for the cysQ gene, selected from the group consisting of
a) a polynucleotide which is at least 70% identical to a polynucleotide coding for a polypeptide which contains the amino acid sequence of SEQ ID no. 2,
b) a polynucleotide which codes for a polypeptide containing an amino acid sequence which is at least 70% identical to the amino acid sequence of SEQ ID no. 2,
c) a polynucleotide which is complementary to the polynucleotides of a) or b), and
d) a polynucleotide containing at least 15 successive nucleotides of the polynucleotide sequence of a), b) or c).

2. The polynucleotide according to claim 1, wherein the polypeptide has transport protein CysQ activity.

3. The polynucleotide according to claim 1, wherein the polynucleotide is a recombinant DNA which can be replicated in coryneform bacteria.

4. The polynucleotide according to claim 1, wherein the polynucleotide is an RNA.

5. The polynucleotide according to claim 3 comprising the nucleic acid sequence as shown in SEQ ID no. 1.

6. The polynucleotide according to claim 3, wherein the DNA, comprises
(i) the nucleotide sequence shown in SEQ ID no. 1, or
(ii) at least one sequence which corresponds to the sequence (i) within the degeneracy region of the genetic code, or
(iii) at least one sequence which hybridizes with the sequence complementary to sequence (i) or (ii).

7. The polynucleotide according to claim 6, further comprising
(iv) functionally neutral sense mutations in (i).

8. The replicable DNA according to claim 5, wherein hybridization is carried out with a stringency corresponding to at most 2× SSC.

9. The polynucleotide sequence according to claim 1, which codes for a polypeptide containing the amino acid sequence shown in SEQ ID no. 2.

10. A Coryneform bacteria in which the cysQ gene is enhanced.

11. The Coryneform bacteria, according to claim 10, wherein the cysQ gene is overexpressed.

12. A method for the fermentative preparation of L-amino acids in coryneform bacteria, comprising:
a) fermenting, in a medium, the coryneform bacteria producing the desired L-amino acid in which at least the endogenous cysQ gene or nucleotide sequences coding therefor are enhanced.

13. The method according to claim 12, further comprising:
b) concentrating the L-amino acid in the medium or in the cells of the bacteria.

14. The method according to claim 13, further comprising:
c) isolating the L-amino acid.

15. The method according to claim 12, wherein the L amino acids are L-lysine, L-cysteine and/or L-methionine.

16. The method according to claim 12, wherein truB gene or nucleotide sequences coding for this gene are overexpressed.

17. The method according to claim 12, wherein additional genes of the biosynthesis pathway of the desired L-amino acid are enhanced in the bacteria.

18. The method according to claim 12, w wherein bacteria in which the metabolic pathways which reduce the formation of the desired L-amino acid are at least partly eliminated are employed.

19. The method according to claim 12, wherein a strain transformed with a plasmid vector is used and the plasmid vector bears the nucleotide sequence coding for the cysQ gene.

20. The method process according to claim 12, wherein the expression of the polynucleotide(s) coding for the cysQ gene is enhanced.

21. The method process according to claim 20, wherein the expression of the polynucleotide(s) coding for the cysQ gene is overexpressed.

22. The method according to claim 12, wherein the catalytic properties of the polypeptide for which the polynucleotide cysQ codes are increased.

23. The method according to claim 12, wherein the bacteria being fermented comprise, at the same time, one or more genes which are enhanced or overexpressed;
wherein the one or more genes is/are selected from the group consisting of:
the dapA gene coding for dihydrodipicolinate synthase,
the gap gene coding for glyceraldehyde-3-phosphate dehydrogenase,
the tpi gene coding for triose phosphate isomerase,
the pgk gene coding for 3-phosphoglycerate kinase,
the zwf gene coding for glucose-6-phosphate dehydrogenase,
the pyc gene coding for pyruvate carboxylase,
the mqo gene coding for malate quinone oxidoreductase,
the lysC gene coding for a feedback resistant aspartate kinase,
the lysE gene coding for lysine export,
the hom gene coding for homoserine dehydrogenase,
the ilvA gene coding for threonine dehydratase or the
ilvA (Fbr) allele coding for a feedback resistant threonine dehydratase,
the ilvBN gene coding for acetohydroxy acid synthase,
the ilvD gene coding for dihydroxy acid dehydratase, and
the zwal gene coding for the Zwal protein.

24. The method according to claim 12, wherein the bacteria being fermented comprise, at the same time, one or more genes which are attenuated; wherein the genes are selected from the group consisting of:
the pck gene coding for phosphoenol pyruvate carboxykinase,
the pgi gene coding for glucose-6-phosphate isomerase,
the poxB gene coding for pyruvate oxidase, and
the zwa2 gene coding for the Zwa2 protein.

25. The method according to claim 12, wherein microorganisms of the Corynebacterium glutamicum type are used.

26. A Coryneform bacteria comprising a vector which includes a polynucleotide according to claim 1.

27. A method for detecting RNA, cDNA and DNA, in order to isolate nucleic acids or polynucleotides or genes which code for the transport protein CysQ or have great similarity with the sequence of the cysQ gene, comprising contacting the RNA, cDNA, or DNA with hybridization probes comprising polynucleotide sequences according to claim 1.

28. The method according to claim 27, wherein arrays, micro arrays or DNA-chips are used.


SEQUENCE LISTING (2 sequences)

SEQ ID NO: 1 - DNA, 2730 bp, Corynebacterium glutamicum
  CDS (1014)..(1769)
tttcattcaaccgactggttaaacgctccgcggaacgcctggaatttacaaacacgatggtcgatttcgccgacatcacctggttgtacacctgctgctcaatgtgtggccaaatcgagccttgagtaggcagcgctgattcgccagtaatccccaacggatcatccatgactagttcaccaatagttgatcccggctcctgaaccggcaaatccgacatgtcttccaccggcacagtgacagtgagatcccactttttctccgcaggtggagccacaatctcaacgggtctgccaccgcccaagaaacccgccaccgtttccaaaggacgcaccgttgcagacaaaccaactcgctgcacaggccgccccacgagcttttccaaacgctccagcgtcaacgccagatgcactccccgtttggttccggccatggcgtggatttcatcgatgatcaccacatcaacatccgaaagggtcgcccccgcttttgaggtcaacatcaaatacgccgactccggagtggtgatcaaaatgtctggaggcttacgcacctgccgggcccgctccgccgatggcgtatcacccgaacgaaccgccacagtgatattgggcacatccaaacccatccgagaggcagtcctcgcaataccggtcaacggtgcacgcagattattttctacatccacgccaagcgctttgagtggggaaatgtagagcactttcactttcccaccacgaacaggcaccggtgttcccgtgtctaaaacctgttgacctgtttgttcagtgagggaatctaacgcccacaaaaacgcagccaaggttttaccactaccggtcggcgccaccacgagggcattcttccccttagataccgcctcccacgttccctcctgaacaggggtcggagatgcaaagacatcccggaaccactccgctacttgaggtcggaatcgggaaagaatgcttttagccatgccttaatgtaaccaaacatctagaattgagaacatgactgctcagattgatgattcgatcctcacccatcgtctcgcccaaggcaccggagaaatcctcaaaggtgtccgcaatgttggggtgttaagaggtcggaatctcggtgatgccggcgacgaactcgcacaaagttggattgctcgagtgttggagcagcaccgccccaacgatggattcctgtctgaagaagccgccgacaacccagaccgcctatccaaggaccgcgtgtggatcatcgatcccctcgacggcaccaaagaattcgccaccggccgccaggactgggcagtacacatcgcactggtagaaaacggtgttcccacccacgccgctgttggcctccccgaccttggcgtggtgttccactccgctgatgcccgcgccgtgactggcccttactccaaggtcatcgccatctcccacaaccgcccaccaaaggttgctctatcttgcgcagagcagctcggctttgaaaccaaggcccttggatccgcaggcgctaaagcaatgcacgttctcctcggtgactacgacgcctacatccacgccggcggccaatacgagtgggattccgcagcaccagtcggcgtctgcaaggcagcaggcttgcactgctccaggctcgacggttccgagctgacctacaacaacaaagacacctacatgccagacatcttgatctgtcgccctgaacttgcagatgaacttctcgagatgtgcgcgaagttctacgaggagaatggaacttactaacgctgttatgatgacggcatgactgttccaacgccttatgaagaccttcttcggaagattgctgaagaagggtcccacaaggacgaccgcaccggcaccggcactacttctttattcggacaacaaatccgctttgatctcaatgaaggttttccccttctgaccaccaagaaggtccatttccactctgttgtgggtgagcttttgtggttccttcagggggattccaacgtcaaatggctgcaggataacaacatccgcatttggaatgaatgggcagatgaggacggcgagctgggccctgtttatggtgtccagtggcgttcttggccaacccctgatggtcgtcacattgaccagatctcaggtgctttagaaactctgcgaaacaaccctgattcacgtcgcaatattgtctcggcgtggaatgtttccgagcttgaaaacatggctcttcccccttgtcacttgcttttccagctctatgtcgccgatggcaaactgtcttgccagctctaccagcgttctgcggacatgttcctgggtgtgcctttcaacatcgcatcttatgcactgctcacccacatgtttgcccagcaggcaggcttggaagtcggcgagttcatttggactggcggcgactgccacatttatgacaaccacaaggaacaggtcgcggagcagctgagccgcgaagctcgcccctaccccaccttggagctcaacaaggcagcgtccatgtttgagtacagcttcgatgacatcaccgtgtccggctacgatccacacccattgatccgcggcaaggtcgccgtatgatcggtgcgatttgggcacaaggccgcgacggcatcatcggcgacggcaccgacatgccctggcacatcccggaagacctcaaacacttcaagaaaaccaccatgggccagccggtcatcatgggtcgtcgcacgtggga

SEQ ID NO: 2 - protein, 252 aa, Corynebacterium glutamicum
MTAQIDDSILTHRLAQGTGEILKGVRNVGVLRGRNLGDAGDELAQSWIARVLEQHRPNDGFLSEEAADNPDRLSKDRVWIIDPLDGTKEFATGRQDWAVHIALVENGVPTHAAVGLPDLGVVFHSADARAVTGPYSKVIAISHNRPPKVALSCAEQLGFETKALGSAGAKAMHVLLGDYDAYIHAGGQYEWDSAAPVGVCKAAGLHCSRLDGSELTYNNKDTYMPDILICRPELADELLEMCAKFYEENGTY